(19) United States
(12) Patent Application Publication
(10) Pub. No.: US 2009/0177218 A1
(43) Pub. Date: Jul. 9, 2009

(54) ULTRASONIC CUTTING TOOL

(76) Inventors: Michael John Radley Young, South Devon (GB); Stephen Michael Radley Young, South Devon (GB)

Correspondence Address: CARTER, DELUCA, FARRELL & SCHMIDT, LLP, 445 BROAD HOLLOW ROAD, SUITE 420, MELVILLE, NY, 11747, US

(21) Appl. No.: 12/162,817

(22) PCT Filed: Jan. 31, 2007
(86) PCT No.: PCT/GB2007/000326

§ 371 Date: Jul. 31, 2008

(30) Foreign Application Priority Data
Jan. 31, 2006 (GB) 0601881.6

(51) Int. Cl. A61B 17/32

(52) U.S. Cl. 606/169

(57) ABSTRACT

The tool's elongate waveguide is operatively connected at a proximal end to a source of ultrasonic vibrations. Cutting and/or coagulating blades are mounted to a distal end of the waveguide. These blades act generally within an operative plane. A handle is mounted to the proximal end of the waveguide and is provided with a re-aligner, operable with a fingertip of a hand grasping the handle, to realign the operative plane of the blades to a preferred direction.

The present invention relates to an ultrasonic surgical tool, such as an ultrasonic laparoscopic tool for cutting soft body tissues. More particularly, but not exclusively, it relates to such a tool that may more easily be aligned to make a cut in a desired plane.

Ultrasonically vibrated cutting tools have proven of major benefit for surgery, particularly laparoscopic surgery (so-called “keyhole” surgery). An elongate, narrow surgical tool, usually together with a fibre-optic viewing system, is introduced through a small incision into a patient's body and steered to an exact region of tissue requiring surgery. Not only do ultrasonically-vibrated tools cut only when ultrasonic energy is selectably applied, but they may easily be adapted to cauterise tissue as they cut. Thus, blood vessels may be both severed and sealed in one operation, for example, significantly reducing bleeding. Such haemostatic cutting is of particular benefit in laparoscopic surgery, where visibility is at a premium.

One particularly useful geometry for a cutting and/or coagulating tool comprises an elongate, ultrasonically vibrated waveguide having a blade member at its distal end and a moveable jaw member, isolated from the ultrasonic vibrations, which may be moved controllably towards contact with the waveguide, trapping tissue to be cut or coagulated therebetween. A good example of such a mechanism, in which the jaw member is pivotably moved towards and away from the waveguide, is disclosed in our UK Patent No. 2333709.

A feature of such jaw mechanisms is that the jaw mechanism usually opens and closes in a particular plane. Thus, to cut an element of tissue in a desired direction, the jaws must be offered up thereto in substantially the right alignment. Most such tools are steered and operated using a handpiece at a proximal end of the tool. A user may thus need to rotate his or her wrist, arm and even shoulder through significant angles, in order to align the distal jaw mechanism correctly with tissue to be treated. Some tools have been proposed in which the relative alignment of the proximal handpiece and the distal jaws may be adjusted to one of a limited number of predetermined angles. This may ease the problem slightly, but such tools require two-handed operation to make the adjustment, which may well be unacceptable for a surgeon partway through a complex and delicate procedure. In any case, the user will still wish to rotate the tool to the exact angle required, which is likely to be between the predetermined angles available for the tool.

There is hence a need for an ultrasonic cutting tool that may more readily be presented to tissue to be treated in a desired alignment, without requiring major bodily contortions from the user.

It is hence an object of the present invention to provide an ultrasonic cutting and/or coagulating tool that obviates the above disadvantages and permits a user readily to present an operative element of the tool to tissue to be treated in a desired alignment without needing to realign a handpiece thereof.

According to the present invention, there is provided an ultrasonic surgical tool comprising elongate waveguide means operatively connected or connectable at a proximal end to a source of ultrasonic vibrations, cutting and/or coagulating means mounted to a distal end of the waveguide means and acting generally within an operative plane, and manually graspable handle means mounted adjacent the proximal end of the waveguide means and provided with means, operable with a fingertip of a hand grasping the handle means, to realign said operative plane of the cutting and/or coagulating means to a preferred direction.

Preferably, said realignment means rotates the cutting and/or coagulating means about a longitudinal axis of the waveguide means.

Advantageously, said longitudinal axis is included in said operative plane.

The realignment means may rotate the waveguide means and the cutting and/or coagulating means together about said longitudinal axis.

Preferably, the realignment means comprises means of mechanical advantage to increase a rotational torque imposable on the cutting and/or coagulating means.

Advantageously, said means of mechanical advantage comprises gearing means.

Said gearing means may provide a mechanical advantage of between 1.5 to 1 and 3 to 1, optionally of around 2 to 1.

Preferably, the realignment means comprises wheel means, contactable and rotatable by a fingertip of a hand grasping the handle means.

The wheel means may be rotatable about an axis extending transversely to the longitudinal axis of the waveguide means, optionally extending substantially orthogonally thereto.

The gearing means may then comprise bevel gear means.

Alternatively, the wheel means may be rotatable about an axis extending generally parallelly to the longitudinal axis of the waveguide means.

The wheel means may be provided with grip means adapted for a fingertip to engage therewith.

Preferably, the realignment means is selectably relocatable on the handle means to suit a fingertip of a particular user.

Advantageously, the realignment means is mounted to a portion of the handle means that is selectably moveable relative to a remainder thereof.

Said portion may be rotatable, optionally about the longitudinal axis of the waveguide means.

In a preferred embodiment, the waveguide means is connected or connectable to a source of torsional mode ultrasonic vibrations.

Advantageously, the cutting and/or coagulating means comprises ultrasonically-vibratable blade means mounted to the distal end of the waveguide means.

The cutting and/or coagulating means may then further comprise a pivotably moveable jaw member, isolated from ultrasonic vibrations and selectably moveable into and out of operative relationship with the blade means.

The jaw member may then be pivoted towards the blade means to engage tissue to be cut and/or cauterised therebetween.

The jaw member thus pivots within said operating plane.

The operating plane may extend away from a cutting edge of the blade means.

In an optional embodiment, the jaw member simultaneously pivots and rotates relative to the longitudinal axis as it moves into and out of operative relationship with the blade means.

A portion of the jaw member's motion immediately adjacent said operative relationship then extends substantially within said operating plane.

Preferably, the handle means of the tool comprises manually operable control means adapted so to move the jaw member.

Advantageously, said control means is operable with the same hand as the realignment means.

The control means may be adjustable to suit a particular user's hand.

Means may further be provided selectably to activate the source of ultrasonic vibrations.

Optionally, said activation means may be mounted to the handle means.

Alternatively, said activation means may comprise pedal means disposed remotely from the handle means.

The waveguide means may be surrounded along a majority of its extent by shroud means isolated from the ultrasonic vibrations.

The jaw member may be pivotably mounted to the shroud means.

An embodiment of the present invention will now be more particularly described by way of example and with reference to the accompanying drawings, in which:

FIG. 1 is a schematic cross-sectional side elevation of a handpiece of a tool embodying the present invention;

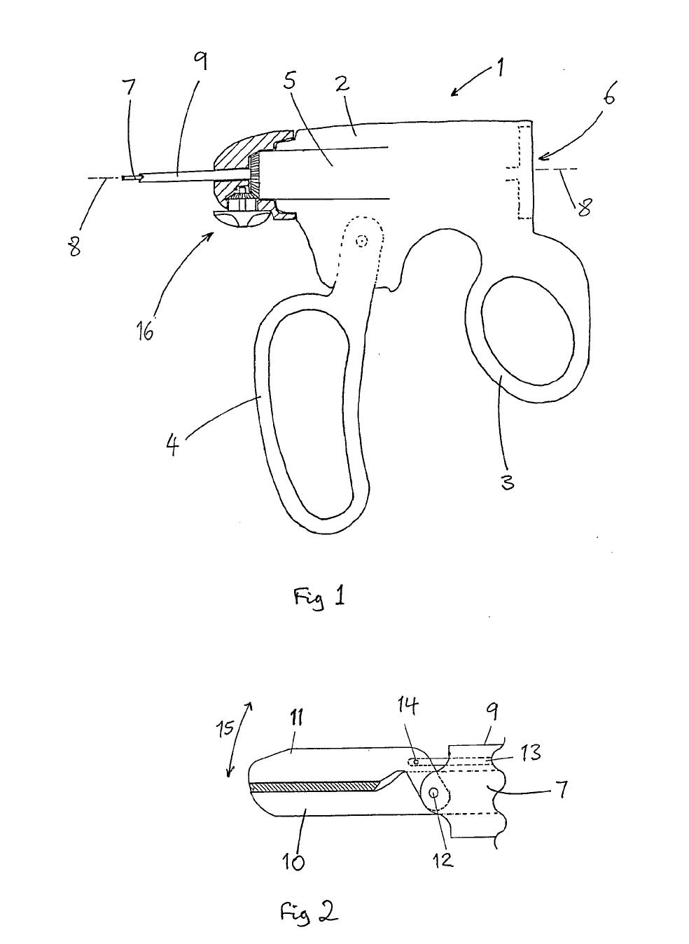

FIG. 2 is a scrap side elevation of a distal jaw mechanism usable with the tool shown in FIG. 1;

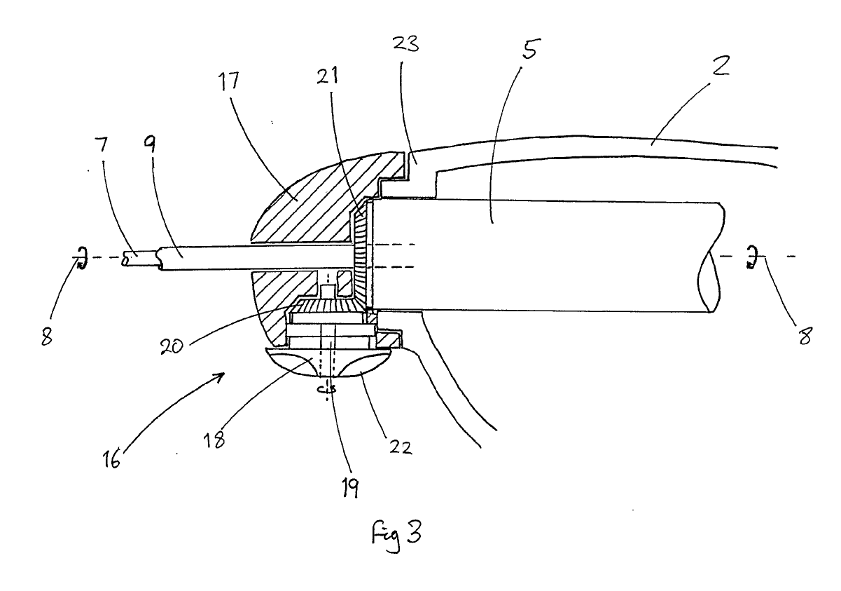

FIG. 3 is a schematic cross-sectional side elevation of a rotation control mechanism of the tool shown in FIG. 1;

FIG. 4 is a plan view from below of the rotation control mechanism shown in FIG. 3;

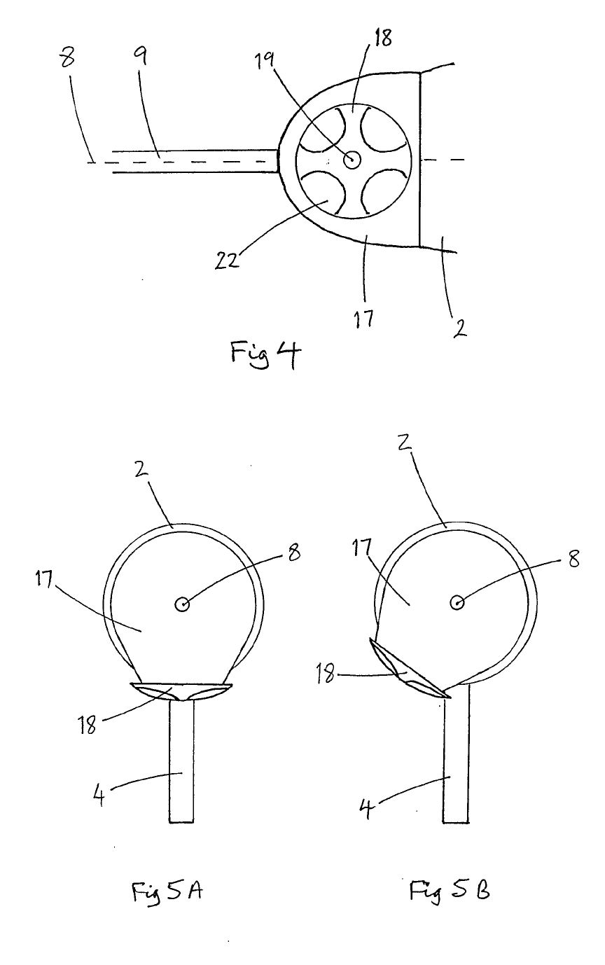

FIGS. 5A and 5B are schematic distal end elevations of the handpiece shown in FIG. 1, with the rotation control mechanism in two alternative dispositions;

FIGS. 6A and 6B are schematic side elevations of a distal jaw mechanism of the tool shown in FIG. 1, in operation;

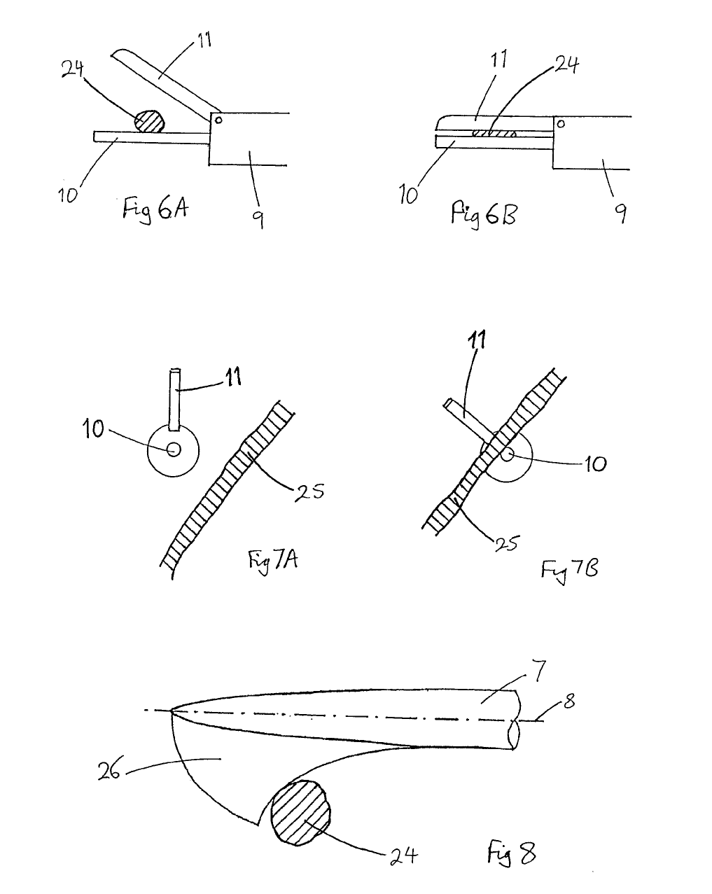

FIGS. 7A and 7B are schematic distal end elevations of the jaw mechanism of FIGS. 6A and 6B, in operation;

FIG. 8 is a side elevation of an alternative distal operative element for the tool shown in FIG. 1.

Referring now to the figures, and to FIG. 1 in particular, an ultrasonic surgical tool 1 comprises a handpiece 2, which is held in one hand of a surgeon or other user. In this case, the handpiece 2 is provided with a thumb ring 3 and a finger bow 4, here adapted to receive a middle, ring and little finger of the surgeon's hand. The finger bow 4 is pivotably mounted to the handpiece 2 so that it may be moved towards and away from the static thumb ring 3, and is operatively linked to a jaw operating mechanism 5 of the tool 1 (details omitted for clarity), the function of which is described below.

A generator for ultrasonic vibrations (not shown) is detachably connectable to the handpiece 2 through an aperture 6 in its proximal end. The particular tool 1 shown is intended for use with torsional mode ultrasonic vibrations, although the present invention is equally applicable to tools employing longitudinal mode ultrasonic vibrations.

An elongate narrow titanium waveguide 7 extends from a distal end of the handpiece 2. The waveguide 7 is operatively connected to the ultrasonic generator and transmits ultrasound vibrations therefrom to an operative element of the tool 1 (e.g. as shown in FIG. 2), mounted to its distal end. The elongate waveguide 7 defines a longitudinal axis 8 of the tool 1. A cylindrical shroud member 9 extends coaxially around the waveguide 7. The shroud member 9 is isolated from the ultrasound vibrations.

FIG. 2 shows one of many operative elements that may be mounted at a distal end of the tool 1. A blade member 10 is mounted to a distal end of the waveguide 7 and is thus ultrasonically vibratable. A jaw member 11 is pivotably mounted to a distal end of the shroud member 9 at a first pivot point 12 and is thus isolated from ultrasonic vibrations. A control rod 13 extends from the jaw operating mechanism 5 of the handpiece 2, within the shroud member 9, to a second pivot point 14 on the jaw member 11.

When the surgeon moves the finger bow 4 towards the thumb ring 3, the jaw operating mechanism 5 causes the control rod 13 to urge the jaw member 11 to pivot into contact with the blade member 10. A reverse motion of the finger bow 4 causes the jaw member 11 to pivot away from the blade member 10.

Tissue to be cut is clamped between the jaw member 11 and the blade member 10, and the waveguide 7 and blade member 10 are then ultrasonically vibrated. The tissue is severed and body fluids such as blood therein are coagulated, providing a neat, controllable, haemostatic cut. (Note: several other mechanisms are known for turning scissor-like handpiece movements into pivoting jaw movements and the invention is not limited to that shown).

Clearly, the jaw member 11 pivots only within a plane shown by arrows 15. When the tool 1 is used in a laparoscopic procedure, it is inserted into the body through a very small incision, usually accompanied by a fibre-optic endoscope arrangement so that the surgeon can view, clamp and cut the target tissue. If this tissue is not aligned with the plane 15 of the jaws, the surgeon must rotate the whole tool 1 about the longitudinal axis 8 until the plane 15 of the jaws is correctly aligned for the required cut. This may lead to the surgeon having to contort his or her wrist, arm, shoulder and occasionally even torso to rotate the tool 1 appropriately. This is clearly highly inconvenient, potentially fatiguing and may interfere with the surgeon's fine control over a cutting procedure. The tool 1 of the present invention is hence provided with a rotation control mechanism 16, as shown generally in FIG. 1 and in more detail in FIG. 3, to overcome this problem.

A body 17 of the rotation control mechanism 16 is mounted to a distal end of the handpiece 2, such that the shroud member 9, waveguide 7 and longitudinal axis 8 pass therethrough. A control wheel 18 is rotatably mounted to the body 17 by means of an axle 19. A first bevel gear 20, also mounted to the axle 19, engages with a larger second bevel gear 21, connected to the jaw control mechanism 5. The axle 19 is rotatable about an axis that intersects at right angles with the longitudinal axis 8, while the second bevel gear 21 is rotatable about the longitudinal axis 8 itself.

The control wheel 18 is provided with four dished recesses 22, dimensioned to receive a fingertip of a hand of a user. The user extends his forefinger to contact the control wheel 18, preferably using a convenient recess 22, and rotates the control wheel 18 on its axle 19. This rotates the first bevel gear 20, which engages with and rotates the second bevel gear 21, and hence the jaw control mechanism 5. This is in turn linked to the shroud member 9 and the waveguide 7, which rotate about the longitudinal axis 8, hence also rotating the plane 15 of the jaw member 11 and the blade member 10. The surgeon may thus “dial” a desired angular alignment of the distal jaw mechanism simply by fingertip rotation of the control wheel 18.

The bevel gears 20, 21 are so relatively dimensioned as to provide a mechanical advantage of between 1.5:1 and 3:1, typically 2:1. Thus, if the control wheel 18 is rotated through 360°, the second bevel gear 21, and hence the distal jaw mechanism, will rotate through only 180°, but the surgeon need only exert half the required turning torque with his forefinger.

The location of the control wheel 18 on an underside, in use, of the body 17 of the rotation control mechanism 16 (as shown in FIG. 4), makes it relatively convenient for a forefinger of a hand grasping the finger bow 4 with some or all of the remaining fingers. However, it is already known that surgical tools with such scissor-like grips are both more controllable and more comfortable if a surgeon may adapt them to a preferred configuration of grip. Our UK Patent No. 2348390 discloses exchangeable finger bows 4, thumb rings 3 and the like, which allow a surgical tool of this type to be adapted for left-handed or right-handed users, for different finger sizes and even for personal preferences such as a number of fingers to be accommodated within a finger bow. To make the tool 1 of the present invention more convenient for a range of users, the location of the control wheel 18 is therefore adjustable.

The body 17 is mounted to a distal shoulder 23 of a casing of the handpiece 2. It may be withdrawn therefrom a small distance distally of the tool 1, rotated about the longitudinal axis 8 and replaced. Thus, as shown in FIGS. 5A and 5B, prior to use of the tool 1, the control wheel 18 may be positioned at an angle, relative to the finger bow 4, that is most comfortable and convenient for the user's forefinger. The configuration shown in FIG. 5B is most suitable for the handpiece 2 to be operated with a user's right hand, for example.

FIGS. 6A, 6B, 7A and 7B illustrate the operation of the tool I on an element 24 of body tissue. The surgeon brings the distal jaw mechanism up to the tissue 24 (FIG. 6A), placing the blade member 10 and jaw member 11 on opposite sides thereof. Using the finger bow 4 and thumb ring 3, he or she brings the jaw member 11 down onto the blade member 10, trapping the tissue 24. The ultrasound generator is then activated (this may be performed with a foot-activated pedal or with a switch located on the handpiece 2), causing intense torsional mode ultrasonic vibrations in the blade member 10 which sever and cauterise the tissue 24. The finger bow 4 and thumb ring 3 are then separated, opening the jaw mechanism for a subsequent cut.

Where, as in FIG. 7A, the jaw member 11 is not aligned conveniently to catch and clamp an element of tissue (such as a blood vessel 25), the surgeon, as a first step, “dials” the control wheel 18 with his or her forefinger until the jaw member 11 has been rotated into a better alignment, as in FIG. 7B, whereupon he or she positions the jaw mechanism around the vessel 25 and proceeds as described above.

Tools with jaw mechanisms are not the only ones to benefit from rotation of their operative distal elements, as described above. FIG. 8 shows a hooked blade 26, as disclosed in our UK Patent No. 2365775. This is mounted to a distal end of the waveguide 7, and is used by disposing a proximally-oriented edge of the hooked blade 26 in contact with the tissue 24 to be cut. The waveguide 7 is then vibrated, preferably with torsional mode ultrasonic vibrations, and the blade 26 is drawn gently in a proximal direction, severing the tissue 24. This tool, too, has a defined plane in which it will cut, which may need to be rotated to operate on a particular piece of tissue. Thus, a rotation control mechanism 16, incorporated in the tool's handpiece so as to permit rotation of the hooked blade 26 controllably about the longitudinal axis 8 of the waveguide 7, will be of significant benefit in this case also.

1. An ultrasonic surgical tool comprising elongate waveguide means operatively connectable at a proximal end to a source of ultrasonic vibrations, cutting and/or coagulating means mounted to a distal end of the waveguide means and acting generally within an operative plane, manually graspable handle means mounted adjacent the proximal end of the waveguide means, and realignment means, operable with a fingertip of a hand grasping the handle means, to realign said operative plane of the cutting and/or coagulating means to a preferred direction.

2. A tool as claimed in claim 1, wherein said realignment means rotates the cutting and/or coagulating means about a longitudinal axis of the waveguide means.

3. A tool as claimed in claim 2, wherein said longitudinal axis is included in said operative plane.

4. A tool as claimed in claim 3, wherein the realignment means rotates the waveguide means and the cutting and/or coagulating means together about said longitudinal axis.

5. A tool as claimed in claim 1, wherein the realignment means comprises means of mechanical advantage, such as gearing means, to increase rotational torque imposable on the cutting and/or coagulating means.

6. A tool as claimed in claim 2, wherein the realignment means comprises wheel means, contactable and rotatable by said fingertip of said hand grasping the handle means.

7. A tool as claimed in claim 6, wherein the wheel means is rotatable about an axis extending transversely to the longitudinal axis of the waveguide means, optionally extending orthogonally thereto.

8. A tool as claimed in claim 6, wherein the wheel means is rotatable about an axis extending generally parallelly to the longitudinal axis of the waveguide means.

9. A tool as claimed in claim 1, wherein the realignment means is selectably relocatable on the handle means to suit a fingertip of a particular user.

10. A tool as claimed in claim 2, wherein the realignment means is mounted to a portion of the handle means that is selectably moveable relative to a remainder thereof.

11. A tool as claimed in claim 10, wherein said portion may be rotatable, optionally about the longitudinal axis of the waveguide means.

12. A tool as claimed in claim 2, wherein the waveguide means is connected or connectable to a source of torsional mode ultrasonic vibrations.
